(19) United States
(12) Patent Application Publication
(10) Pub. No.: US 2025/0281669 A1
(43) Pub. Date: Sep. 11, 2025

(54) Method for Preparing Skull Flap by Photo-Curing 3D Printing

(71) Applicant: Beijing Tiantan Hospital, Capital Medical University, Beijing (CN)

(72) Inventors: Yilong WANG, Beijing (CN); Yuchun GU, Beijing (CN); Weiqi CHEN, Beijing (CN); Bowen CHI, Beijing (CN); Lida WU, Beijing (CN); Yongjun WANG, Beijing (CN); Lingling JIANG, Beijing (CN)

(21) Appl. No.: 18/811,750

(22) Filed: Aug. 21, 2024

(30) Foreign Application Priority Data
Mar. 8, 2024 (CN) 202410267661.4

(51) Int. Cl. A61L 27/22; A61L 27/04; A61L 27/12; A61L 27/20; A61L 27/36; A61L 27/54; B33Y 10/00; B33Y 50/00; C08L 5/04; C08L 5/08; C08L 89/06

(52) U.S. Cl. CPC A61L 27/222; A61L 27/047; A61L 27/12; A61L 27/20; A61L 27/3633; A61L 27/54; B33Y 10/00; B33Y 50/00; C08L 5/04; C08L 5/08; C08L 89/06; A61L 2300/252; A61L 2300/414

(57) ABSTRACT

Provided is a method for preparing a skull flap by photo-curing 3D (three dimensions) printing, including the following steps: (1) constructing a 3D printing model: generating a corresponding 3D model based on a cranial scan data of a patient; (2) preparing a 3D printing ink: weighing a photo-crosslinking hydrogel, mixing the photo-crosslinking hydrogel and a nanoclay solution to obtain a mixed system, and then adding a photoinitiator and a photoresist into the mixed system to obtain the 3D printing ink; and (3) conducting photo-curing 3D printing: subjecting the 3D printing ink to ultrasonic mixing, then filling into an ink tank of a photo-curing 3D printer, transferring the corresponding 3D model into the photo-curing 3D printer, and subjecting the corresponding 3D model to printing to obtain the skull flap; wherein the nanoclay solution comprises an extracellular matrix component.

### CROSS REFERENCE TO RELATED APPLICATION

This application claims the benefit and priority of Chinese Patent Application No. 2024102676614 filed with the China National Intellectual Property Administration on Mar. 8, 2024, the disclosure of which is incorporated by reference herein in its entirety as part of the present application.

### TECHNICAL FIELD

The present disclosure relates to the technical field of medical materials, and in particular to a method for preparing a skull flap by photo-curing 3D (three dimensions) printing.

### BACKGROUND

Existing 3D printing technology for skull is mainly printing of metal materials. However, most of the special materials for medical device production require strict in vivo and in vitro biological evaluation to prevent the occurrence of various biological risks. Metal materials are not biologically active and have poor thermal and electrical conductivity, which are the main deficiencies of 3D-printed skulls. Furthermore, due to the different molding principles of 3D printing, printing materials need to be liquefied, silken, or powdered, and then rejoined after printing. Therefore, the 3D printing also shows special requirements for raw materials. The high difficult development and the long evaluation cycle of the printing materials constitutes the difficulties and core for the research and development on 3D printing of auxiliary medical devices.

Among the currently commonly-used 3D printing methods, micro-extrusion 3D bio-printing has become a reliable process for 3D printing of bone repair scaffolds due to its simple operation, high efficiency, and relatively low cost. However, this printing process generally involves organic solvents or high temperature, which results in the loss of biological activities of the scaffold. Moreover, such printing process also shows low precision and thus could easily lead to relatively large pores in the printed product, which are not conducive to cell deposition and adhesion. Although it is a common printing process for printing single organic biological materials, it is impossible to achieve uniform dispersion of printing materials when printing nano-scale materials due to inherent defects in this printing process.

### SUMMARY

To solve the above problems, the present disclosure provides a method for preparing a skull flap by photo-curing 3D printing. In the method, methacrylated gelatin (GelMA) and nanoclay are used as printing raw materials, which have desirable photo-crosslinking property, high bio-compatibility, and mechanical strength that meets the supporting function; and ultrasonic mixing before printing makes the 3D printing ink more uniform, thereby resulting in that the whole structure to be printed has a uniform composition, which is beneficial for skull repair.

Additional features and advantages of the present disclosure will be set forth in part in the specification below, and in part will be apparent from the specification, or may be learned by practice of the present disclosure. The objects and other advantages of the present disclosure will be realized and obtained by means of the elements and combinations particularly pointed out in the claims appended to the specification.

To achieve the above advantages, the present disclosure provides a method for preparing a skull flap by photo-curing 3D printing, including the following steps:
(1) constructing a 3D printing model: generating a corresponding 3D model based on a cranial scan data of a patient;
(2) preparing a 3D printing ink: weighing a photo-crosslinking hydrogel, mixing the photo-crosslinking hydrogel and a nanoclay solution to obtain a mixed system, and then adding a photoinitiator and a photoresist into the mixed system to obtain the 3D printing ink; and
(3) conducting photo-curing 3D printing: subjecting the 3D printing ink to ultrasonic mixing, then filling into an ink tank of a photo-curing 3D printer, transferring the corresponding 3D model into the photo-curing 3D printer, and subjecting the corresponding 3D model to printing to obtain the skull flap; where the nanoclay solution includes an extracellular matrix component.

In the present disclosure, the photo-crosslinking hydrogel mainly composed of organic components and the nanoclay mainly composed of inorganic components are used as the main raw materials of a photo-curing printing ink. The nanoclay is evenly dispersed inside the photosensitive hydrogel through ultrasonic mixing, while adhesive properties of the hydrogel could enable the nanoclay to be evenly dispersed in different locations of the bone flap during the printing. Furthermore, the ultrasonic mixing of the printing ink before printing is more conducive to the uniform dispersion of the nanoclay solution in an overall structure of the skull flap.

In order to facilitate the conversion of scan data into 3D printing models, in some embodiments, the cranial scan data of the patient in step (1) is obtained through computed tomography (CT) scanning or magnetic resonance imaging (MRI).

In order to obtain a 3D printing ink with a better printing performance, in some embodiments, the 3D printing ink in step (2) is prepared by weighting 5 wt % to 20 wt % of the photo-crosslinking hydrogel, mixing the 5 wt % to 20 wt % of the photo-crosslinking hydrogel and 1 wt % to 4 wt % of the nanoclay solution to obtain the mixed system, and then adding 0.5 wt % to 5 wt % of the photoinitiator and 0.001 wt % to 3 wt % of the photoresist into the mixed system. It has been verified through experiments that the skull flap printed within the above component concentration range is more precise and has a well-balanced mechanical strength such as hardness and toughness, allowing the skull flap to better protect and repair the intracranium.

In order to obtain faster gel formation speed and better mechanical properties, in some embodiments, the photo-crosslinking hydrogel is at least one selected from the group consisting of methacrylated gelatin (GelMA), methacryloyl sodium alginate (AlgMA), and methacrylated hyaluronic acid (HAMA); and in further embodiments, the photo-crosslinking hydrogel is the GelMA. The GelMA is injectable, gels quickly, shows desirable mechanical properties and bio-compatibility, and thus is suitable for customized bio-printing.

In order to obtain the mechanical strength of the photo-crosslinking hydrogel in the photo-curing composition without affecting the photosensitive properties of the hydrogel, in some embodiments, the nanoclay in the nanoclay solution is at least one selected from the group consisting of hydroxyapatite, tricalcium phosphate, and lithium magnesium silicate; and in further embodiments, the nanoclay is the lithium magnesium silicate. The lithium magnesium silicate has a nano-scale 3D structure, shows excellent physical and chemical properties as well as bio-compatibility, exhibits certain antibacterial properties, and could promote cell proliferation and differentiation.

To form a support on the skull flap and connect the cells together, an extracellular matrix component is added into the nanoclay solution. In some embodiments, the extracellular matrix component includes at least one selected from the group consisting of a calcium phospholipid component, collagen protein, and glycosaminoglycan, which carries a large number of signaling molecules that actively participate in controlling cell growth, polarity, shape, migration, and metabolic activities.

In order to enable the photo-curing composition to promote the differentiation of bone cells and blood vessels and the reconstruction of the neuroimmune environment, in some embodiments, the photo-curing composition further includes a chemokine. The chemokine could enter the skull environment along with the photo-curing composition to promote cell differentiation and neuroimmune environment reconstruction during the skull repair.

In some embodiments, the chemokine is at least one selected from the group consisting of a ciliary neurotrophic factor (CNTF) neural factor, a vascular endothelial growth factor (VEGF) vascular factor, and a bone morphogenetic protein 2 (BMP-2) bone repair factor. The CNTF neural factor could promote the synthesis of neurotransmitters and the axonal growth of certain neuron groups in the nervous system, and could also be expressed in bone surface cells. The VEGF vascular factor has the effects such as promoting increased vascular permeability, extracellular matrix degeneration, vascular endothelial cell migration, proliferation, and blood vessel formation. The BMP2 bone repair factor could stimulate DNA synthesis and cell replication, thereby promoting the directional differentiation of mesenchymal cells into osteoblasts. The BMP-2 bone repair factor is also a main factor inducing bone and cartilage formation in vivo, and is expressed during limb growth, endochondral ossification, early fracture, and cartilage repair, and plays an important role in the embryonic development and regeneration repair of bones. Further, in order to enable the photo-curing composition to better promote the repair and differentiation of bone cells and the generation of peripheral blood vessels in the skull defect after entering the skull, in some embodiments, the chemokine is composed of the CNTF neural factor, VEGF vascular factor, and BMP-2 bone repair factor, which work synergistically in the skull to promote skull repair.

For uniform dispersion of nanoclay in bioink (i.e., 3D printing ink), in some embodiments, the ultrasonic mixing in step (3) is conducted at a power of 1,000 W to 1,300 W, preferably 1,200 W for 0.5 seconds to 1.5 seconds. The ultrasonic mixing within the above parameter range could achieve better dispersion effects.

In order to facilitate printing with the photo-curing 3D printer, the corresponding 3D model in step (3) is cut into multiple two-dimensional layers through model slicing, and then the multiple two-dimensional layers are transferred into the photo-curing 3D printer and subjected to the printing.

### Beneficial Effects:

(1) In the present disclosure, the photo-crosslink hydrogel mainly composed of organic components and the nanoclay mainly composed of inorganic components are used as the main raw materials of photo-curing printing ink, and satisfy the special material requirements of photo-curing 3D printing. Compared with metal materials, the combined material of hydrogel and nanoclay for 3D printing has sufficient mechanical properties to match the native skull while greatly shortening the time for subsequent polishing of the printed skull. Moreover, due to its high bioactivity and soft properties, this 3D printed implant shortens the patient's painful and stressful adaptation period. Furthermore, the printing ink using the nanoclay and hydrogel system improves the printing accuracy of photo-curing 3D printing, so that the printed skull flap is enough to mimic the complex structures of the human meninges and bone marrow cavity. For skull defects, the construction of bionic structures of the meninges and marrow cavity is also highly important for their contribution to the repair of skull defects.

(2) In the present disclosure, a printing ink of nanoclay and hydrogel system is used. The nanoclay is evenly dispersed inside the photosensitive hydrogel through ultrasonic, while adhesive properties of the hydrogel could enable the nanoclay to be evenly dispersed in different locations of the bone flap during the printing. Meanwhile, the ultrasonic mixing of the printing ink before printing is more conducive to the uniform dispersion of the extracellular matrix component existing in the nanoclay solution in an overall structure of the skull flap.

### BRIEF DESCRIPTION OF THE DRAWINGS

To describe the technical solutions in the embodiments of the present disclosure more clearly, the following briefly describes the accompanying drawings required for describing the examples of the present disclosure. Apparently, the accompanying drawings in the following description show merely some embodiments of the present disclosure, and those of ordinary skill in the art may still derive other accompanying drawings from these accompanying drawings without creative efforts.

FIG. 1 shows an appearance of the printed sample prepared in Example 1 of the present disclosure;

FIG. 2 shows a mechanical property test of the printed sample prepared in Example 1 of the present disclosure;

FIG. 3 shows long-term swelling test results of the printed sample prepared in Example 1 of the present disclosure;

FIGS. 4A-4D show the staining of live and dead cells on the printed product prepared in Example 1 of the present disclosure under Live, dead, TD (i.e. no fluorescence), and merge light source channels of a cell transplantation test, respectively;

FIG. 5 shows micro-CT results of the effectiveness of skull defect repair in an animal experiment on the printed sample prepared in Example 1 of the present disclosure;

FIG. 6 shows the Masson staining effect of the skull defect repair in an animal experiment on the printed sample prepared in Example 1 of the present disclosure;

FIG. 7 shows an appearance of the printed sample prepared in Comparative Example 1 of the present disclosure;

FIG. 8 shows a mechanical property test of the printed sample prepared in Comparative Example 1 of the present disclosure;

FIG. 9 shows long-term swelling test results of the printed sample prepared in Comparative Example 1 of the present disclosure;

FIGS. 10A-10D show the staining of live and dead cells on the printed product prepared in Comparative Example 1 of the present disclosure under Live, dead, TD (i.e. no fluorescence), and merge light source channels of a cell transplantation test, respectively;

FIG. 11 shows micro-CT results of the effectiveness of skull defect repair in an animal experiment on the printed sample prepared in Comparative Example 1 of the present disclosure;

FIG. 12 shows the Masson staining effect of the skull defect repair in an animal experiment on the printed sample prepared in Comparative Example 1 of the present disclosure;

FIG. 13 shows an appearance of the printed sample prepared in Comparative Example 2 of the present disclosure;

FIG. 14 shows a mechanical property test of the printed sample prepared in Comparative Example 2 of the present disclosure;

FIG. 15 shows long-term swelling test results of the printed sample prepared in Comparative Example 2 of the present disclosure;

FIGS. 16A-16D show the staining of live and dead cells on the printed product prepared in Comparative Example 2 of the present disclosure under Live, dead, TD (i.e. no fluorescence), and merge light source channels of a cell transplantation test, respectively;

FIG. 17 shows micro-CT results of the effectiveness of skull defect repair in an animal experiment on the printed sample prepared in Comparative Example 2 of the present disclosure;

FIG. 18 shows the Masson staining effect of the skull defect repair in an animal experiment on the printed sample prepared in Comparative Example 2 of the present disclosure; and

FIG. 19 shows a schematic structure of the 3D printing model of the skull flap according to an embodiment of the present disclosure.

### DETAILED DESCRIPTION OF THE EMBODIMENTS

The photo-curing material according to the present disclosure will be described in more detail with reference to the following examples. Apparently, the described examples are some rather than all of the embodiments of the present disclosure. All other embodiments obtained by those of ordinary skill in the art based on the embodiments of the present disclosure without creative efforts should fall within the scope of the present disclosure.

In the description of the embodiments of the present disclosure, it should be noted that all ranges disclosed in the present disclosure will be understood to encompass any and all sub-ranges included therein. For example, a stated range of “2.0 to 13.0” shall be deemed to include any and all subranges that begin with a minimum value of 2.0 or greater and end with a maximum value of 13.0 or less, for example, 2.0 to 6.2, or 3.5 to 10.0, or 5.2 to 7.9. Also, all ranges disclosed herein are also deemed to include the endpoints of the ranges unless expressly stated otherwise. For example, the range “between 4 and 6” or “4 to 6” or “4-6” should generally be considered to include the endpoints 4 and 6.

Likewise, it is to be understood that the phrases and terminology used in the present disclosure are for the purpose of description and should not be regarded as limiting. The use of “including,” “comprising,” or “having” and variations thereof in this disclosure is intended to include the items listed thereafter and their equivalents as well as additional items in an open-ended manner.

As used herein in the specification and claims, the phrase “at least one”, with reference to a list of one or more elements, should be understood to mean at least one element selected from any one or more elements in the list of elements. However, at least one of every element specifically enumerated in the element list is not necessarily included, and any combination of elements in the element list is not excluded.

The term “3D printing” and the like generally describe a variety of solid free-form manufacturing techniques for fabricating three-dimensional articles or objects by: stereolithography, selective deposition, jetting, fused deposition molding, multi-jet molding, digital light processing, gel deposition, as well as other additive manufacturing technologies known in the art or known in the future that use building materials or inks to create three-dimensional objects.

The primary meaning of the term “biocompatibility” when describing hydrogels in the present disclosure is to provide positive and controllable effects on biological and functional structures, including interactions with endogenous tissues or the immune system, supporting appropriate cellular activity, and promoting molecular or mechanical signaling systems. These effects are critical to the biological function of the graft.

The present disclosure provides a method for preparing a skull flap by photo-curing 3D printing, including the following steps:

(1) Construction of 3D printing model:

### 1. Construction of 3D Printing Model

1.1 Patient data acquisition: cranial CT scanning or MRI image data of a patient is obtained.

1.2 Image processing: the cranial CT scanning or the MRI image data is converted into a 3D model by using medical image processing software such as DICOM Viewer. The accurate models of skull defects are generated by adjusting image resolution and selecting regions of interest.

1.3 Design of 3D model: by using computer-aided design (CAD) software, a 3D printing model adapted to the patient's skull defect is designed, in which the microchannel structure is restored as much as possible.

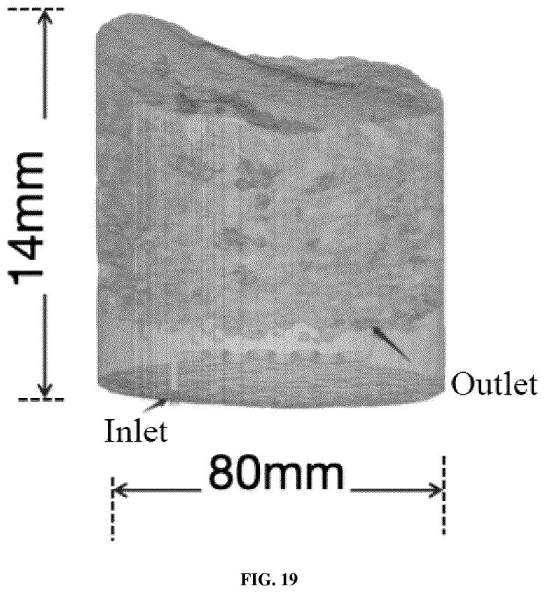

1.4 Model optimization: the model is optimized to ensure a stable and accurate build during the 3D printing while taking into account the support structure and printing direction. The patient's cranial scan data is obtained through CT scanning or MRI, and a corresponding 3D model is generated based on the patient's cranial scan data, as shown in FIG. 19.

### (2) Preparation of 3D Printing Ink:

5 wt % to 20 wt % of the photo-crosslinking hydrogel is weighed and then mixed with 1 wt % to 4 wt % of the nanoclay to be uniform, and 0.5 wt % to 5 wt % of the photoinitiator and 0.001 wt % to 3 wt % of the photoresist are then added thereto to obtain a 3D printing ink, where the nanoclay solution includes an extracellular matrix component.

### (3) Photo-Curing 3D Printing:

3.1 Setting up 3D printing equipment: the 3D printer is configured with desirable parameters for manufacturing organ scaffolds, including printing speed, temperature, and layer height.

3.2 Filling of ink: the 3D printing ink is subjected to ultrasonic mixing at a power of 1,000 W to 1,300 W for 0.5 s to 1.5 s, and then filled into the ink tank of the photo-curing 3D printer and subjected to 3D printing. At the same time, a tea polyphenol solution is used for coating an outer surface of the skull flap. The tea polyphenol coated on the surface of the skull flap could avoid possible water-absorbed swelling of the photo-curing composition prepared by the photo-crosslinking hydrogel in the skull.

3.3 3D printing: a designed 3D model is cut into multiple two-dimensional layers through model slicing, and then the multiple two-dimensional layers are transferred to the photo-curing 3D printer and subjected to, printing to obtain the skull flap.

### (4) Post-Treatment Process:

4.1 Removal of supporting structures: any unnecessary supporting structures are removed from the skull flap after 3D printing is completed.

4.2 Surface modification: necessary surface modifications are conducted on the printed skull flap, such as polishing, cleaning, or chemical treatment to ensure smoothness and bio-compatibility of the surface.

4.3 Packaging: the modified skull flap shall be appropriately packaged to ensure integrity and sterility during storage and transportation prior to product application.

### Extracellular Matrix Component

The extracellular matrix in the present disclosure refers to a complex network composed of a variety of macromolecules around cells in multicellular organisms. The extracellular matrix component includes at least one selected from the group consisting of a calcium phospholipid component, collagen protein, glycosaminoglycan, non-collagen protein, elastin, proteoglycan, and aminoglycan.

### Nanoclay

In the present disclosure, the nanoclay may include nanoparticles of layered mineral silicates such as hydroxyapatite, tricalcium phosphate, and lithium magnesium silicate, may also include nanomaterials such as montmorillonite, bentonite, kaolinite, hectorite, and halloysite that have certain adsorption capabilities and are compatible with a variety of polymer systems, and may also include degradable materials such as calcium silicate, bioglass, akermanite, calcium akermanite, and diopside.

### Photo-Crosslinking Hydrogel

An important feature of biomaterials suitable for bioprinting is the ability to rapidly form in situ and ensure the existence of cells through various cross-linking methods, including physical methods, chemical methods, and photo-crosslinking methods. In the present disclosure, the photo-crosslinking hydrogel refers to a compound containing a photosensitive group that could form a three-dimensional network hydrogel through intramolecular or intermolecular cross-linking under the irradiation of ultraviolet light or visible light. There are two main types of common photo-crosslinking methods. One type is that under the irradiation of ultraviolet light, the photoinitiator undergoes a cleavage reaction to form active free radicals, and a polymer containing vinyl functional groups is initiated to undergo a series of rapid polymerization reactions to form a gel. The other type is that without additional photoinitiator, a polymer containing cross-linkable groups undergoes cross-linking reaction to form a gel. Compared with other cross-linking methods, the photo-crosslinking reaction has mild conditions, fewer by-products and no need of toxic initiators, and its process could be controlled by simply adjusting the intensity, irradiation time, and irradiation range of light. In addition, the photo-crosslinking hydrogel could quickly encapsulate cells and growth factors in situ, thereby improving the survival rate of cells. The presence of growth factors plays an important role in promoting cell proliferation and differentiation, tissue repair, and regeneration. When being synchronously photopolymerized and printed with cells into a three-dimensional hydrogel, the growth factor could improve the viability of the encapsulated cells.

In some embodiments, the photo-crosslinking hydrogel includes GelMA, AlgMA, or HAMA. In further embodiments, the photo-crosslinking hydrogel is the GelMA. It should be noted that when using the GelMA as a photo-curing printing material, it is crucial to control the viscosity of gelatin. A high viscosity of the photo-curing printing material could maintain the fidelity of deposition in 3D printing, and a higher viscosity could usually be obtained by adjusting the concentration of a GelMA solution. However, an excessive concentration of the GelMA may lead to a dense polymer network, inevitably reducing cell activity. Therefore, in some embodiments, the GelMA has a concentration of 5 wt % to 20 wt %, preferably 10 wt % to 15 wt %.

### Chemokine

In the present disclosure, the chemokine refers to a type of small cytokine or signaling protein secreted by cells. The chemokine has the ability to induce directional chemotaxis of nearby reaction cells, such that the human body could promote directional chemotaxis of immune cells when the human body defends and removes foreign bodies such as invading pathogens. The chemokine may include any one of a CNTF neural factor, a VEGF vascular factor, and a BMP-2 bone repair factor, or a mixture of two or more of the above three factors. In some embodiments, the chemokine is a mixture of the CNTF neural factor, the VEGF vascular factor, and the BMP-2 bone repair factor.

### Photoinitiator

In the present disclosure, the photoinitiator refers to an agent capable of initiating photopolymerization. The photoinitiator may include any one of phenyl ketone, phenyl dimethylketone, phenyl trimethylketone, benzoin ether, benzoin bis-ether, α-hydroxy-alkylphenone, and α-alkoxy-alkylphenone, or a mixture of two or more of the above photoinitiators. In some embodiments, the photoinitiator further includes 2-naphthalenesulfonyl chloride and analogs thereof, methoxyacetophenone and analogs thereof, benzoin and analogs thereof, benzyl and analogs thereof, benzophenone, and benzyl dimethyl ketal and analogs thereof.

### Photoresist

In the present disclosure, the photoresist refers to an agent added to inhibit lateral photopolymerization during 3D printing, and may include Sudan Orange G, Sudan I, and Tinuvin 171.

The nanoclay is present in the photo-curing material in the following weight percentages: 1 wt % to 4 wt %; 1 wt % to 3 wt %; 1 wt % to 2 wt %; 2 wt % to 4 wt %; about 3%.

The photo-crosslinking hydrogel is present in the photo-curing material in the following weight percentages: 5 wt % to 20 wt %; 8 wt % to 18 wt %; 10 wt % to 16 wt %; 12 wt % to 18 wt %; 13 wt % to 17 wt %; 15 wt % to 19 wt %; 16 wt % to 18 wt %; about 15%.

The photoinitiator is present in the photo-curing material in the following weight percentages: 0.5 wt % to 5 wt %; 1 wt % to 5 wt %; 2 wt % to 4.5 wt %; 2.5 wt % to 4 wt %; 2.5 wt % to 3.5%; 2.5 to 3 wt %; about 3.5 wt %.

The photoresist is present in the photo-curing material in the following weight percentages: 0.001 wt % to 3 wt %; 0.3 wt % to 2.8 wt %; 0.5 wt % to 2.6 wt %; 1.2 wt % to 2.4 wt %; 1.5 wt % to 2.2 wt %; about 1.5 wt %.

The tea polyphenol is present in the photo-curing material in the following weight percentages: 0.1 wt % to 22 wt %; 1 wt % to 20 wt %; 5 wt % to 18 wt %; 8 wt % to 16 wt %; 10 wt % to 15 wt %; 12 wt % to 14 wt %; about 11 wt %.

The chemokine is present in the photo-curing material at a concentration of: CNTF neural factor (0.1-100 μg/mL), VEGF vascular factor (1-100 μg/mL), and BMP-2 bone repair factor (0.1-200 μg/mL); CNTF neural factor (30-80 μg/mL), VEGF vascular factor (30-80 μg/mL), and BMP-2 bone repair factor (50-180 μg/mL); CNTF neural factor (30-80 μg/mL), VEGF vascular factor (20-80 μg/mL), and BMP-2 bone repair factor (30-180 g/mL); CNTF neural factor (35-60 μg/mL), VEGF vascular factor (38-80 μg/mL), and BMP-2 bone repair factor (50-160 μg/mL); and CNTF neural factor (about 50 μg/mL), VEGF vascular factor (approximately 50 μg/mL), and BMP-2 bone repair factor (about 100 μg/mL).

The present disclosure is further described below with reference to specific examples.

### Example 1

The example 1 provided a 3D printing photo-curing composition for skull repair and a preparation method thereof, where the preparation method was conducted as follows:

Preparation of nanoclay: 0.2006 g of nanoclay was weighed and added into 10 mL of sterile purified water, and then subjected to ultrasonic mixing for 3 min to be uniform so as to obtain a nanoclay solution with a concentration of 2%.

Preparation of a nanoclay solution combined with chemokine: CNTF neural factor (0.1 μg/mL), VEGF vascular factor (1 μg/mL) and BMP2 bone repair factor (0.1 μg/mL) were dissolved in the nanoclay solution, and stored at 37° C. in the dark for later use.

Preparation of a printing photo-curing composition precursor: 0.7506 g of GelMA freeze-dried sample was weighed and mixed with 5 mL of the nanoclay solution with a concentration of 2 wt %, shaken in a centrifuge at a speed of 1,000 rpm for 15 min to obtain the printing photo-curing composition precursor with a GelMA concentration of 15%, and stored at 37° C. in the dark for later use.

Preparation of a printing photo-curing composition: 1 mL of the printing photo-curing composition precursor, 60 μL of a photoinitiator with a concentration of 5 wt %, and 17 μL of a photoresist with a concentration of 3 wt % were mixed to be uniform to obtain a mixture, which was prepared for immediate use and added together with 0.5 mL of a tea polyphenol solution (as an outer surface coating of the printed product) into an ink tank of a photo-curing printer. Printing was conducted by using a projection photo-curing printer, with an exposure time of 6 s for each layer and a light intensity of 20 mW/cm² to obtain a photo-curing print.

### Comparative Example 1

This comparative example was conducted similar to Example 1, except that the chemokine was not added into the photo-curing composition as outer surface coating for printed sample. This comparative example provided a 3D printing photo-curing composition and a preparation method thereof, where the preparation method was conducted as follows:

Preparation of nanoclay: 0.2006 g of nanoclay was weighed and added into 10 mL of sterile purified water, and then subjected to ultrasonic mixing for 3 min to be uniform so as to obtain a nanoclay solution with a concentration of 2%.

Preparation of a printing photo-curing composition precursor: 0.7506 g of GelMA freeze-dried sample was weighed and mixed with 5 mL of the nanoclay solution with a concentration of 2 wt %, shaken in a centrifuge at a speed of 1,000 rpm for 15 min to obtain the printing photo-curing composition precursor with a GelMA concentration of 15%, and stored at 37° C. in the dark for later use.

Preparation of a printing photo-curing composition: 1 mL of the printing photo-curing composition precursor, 60 μL of a photoinitiator with a concentration of 5 wt %, and 17 μL of a photoresist with a concentration of 3 wt % were mixed to be uniform to obtain a mixture, which was prepared for immediate use and added together with 0.5 mL of a tea polyphenol solution (as an outer surface coating of the printed product) into a tank. Printing was conducted by using a projection photo-curing printer, with an exposure time of 6 s for each layer and a light intensity of 20 mW/cm² to obtain a photo-curing print.

### Comparative Example 2

This comparative example was conducted similar to Example 1, except that the tea polyphenol was not added into the photo-curing composition as the outer surface coating of the printed product. This comparative example provided a 3D printing photo-curing composition and a preparation method thereof, where the method was conducted as follows:

Preparation of nanoclay: 0.2006 g of nanoclay was added into 10 mL of sterile purified water, and then subjected to ultrasonic mixing for 3 min to be uniform so as to obtain a nanoclay solution with a concentration of 2%.

Preparation of a nanoclay solution combined with chemokine: CNTF neural factor (0.1 μg/mL), VEGF vascular factor (1 μg/mL) and BMP2 bone repair factor (0.1 μg/mL) were dissolved in the nanoclay solution, and stored at 37° C. in the dark for later use.

Preparation of a printing photo-curing composition precursor: 0.7506 g of GelMA freeze-dried sample was weighed and mixed with 5 mL of the nanoclay solution with a concentration of 2 wt %, shaken in a centrifuge at a speed of 1,000 rpm for 15 min to obtain the printing photo-curing composition precursor with a GelMA concentration of 15%, and stored at 37° C. in the dark for later use.

Preparation of a printing photo-curing composition: 1 mL of the printing photo-curing composition precursor, 60 μL of a photoinitiator with a concentration of 5 wt %, and 17 μL of a photoresist with a concentration of 3 wt % were mixed to be uniform to obtain a mixture, which was prepared for immediate use and added into an ink tank of a photo-curing printer. Printing was conducted by using a projection photo-curing printer, with an exposure time of 6 s for each layer and a light intensity of 20 mW/cm² to obtain a photo-curing print.

The advantages of Example 1 in terms of printing effect were demonstrated through the following tests.

I. Appearance test: visual inspection;

II. Printing effect test: observation was conducted that whether the appearance of the sample printed using the same program was consistent with the program settings;

III. Mechanical properties test:

3.1 Instruments and equipment: Universal testing machine

3.2 Experimental methods

3.2.1 The universal testing machine was turned on and preheated for five minutes.

3.2.2 The software was opened and connected to the universal testing machine, and the testing machine was started. A method was set as compression method, the sample size-compression speed-polar direction were adjusted, and the method was saved.

3.2.3 The sample was placed in the middle of the clamps, the upper clamp was lowered to 1 mm from the upper surface of the material by using a manual control box, the force and displacement were reset to zero through the software, the testing was started to obtain the mechanical performance diagram of the sample.

3.2.4 The curve was observed, the experiment was stopped after the material was broken, the clamp was lifted by using the manual control box, the broken sample was removed and the surfaces of the clamp were cleaned with lint-free cloth and alcohol, and the curve graph and experimental data were saved to the computer.

3.2.5 After the experiment, the software and the universal testing machine were turned off in sequence.

IV. Long-term swelling performance test

4.1 Experimental reagents: PBS solution

4.2 Experimental methods

The newly prepared gel was weighed as G1 and placed in a 12-well plate, 1 mL of sterile water was added to each well to fully immerse the gel sample. After incubation at 37° C., the gel was taken out at 1st, 3rd, 7th, 14th, and 21st days separately, and its long-term swelling rate was calculated. The gel was dried with dust-free paper, placed on a glass slide and weighed to obtain a wet weight G2. The swelling rate of the gel was calculated according to the following formula and a curve was plotted to obtain the swelling test chart.

$$Swelling rate = \frac{G 2 - G 1}{G 1} \times 100$$

V. Cell bio-compatibility test:

5.1 Principle

Calcein acetoxymethyl ester (Calcein-AM) is a cell staining reagent for fluorescent labeling of living cells, and emits green fluorescence (Ex=490 nm, Em=515 nm). The Calcein-AM introduces an acetyl methoxymethyl ester (AM) group on the basis of traditional Calcein to increase hydrophobicity, allowing the Calcein-AM to easily penetrate living cell membranes. Once inside the cell, the Calcein-AM (which itself does not fluoresce) is cleaved by intracellular esterases to form the membrane-impermeable polar molecule Calcein, and then retained in the cell and emits strong green fluorescence. Propidiumiodide (PI) could not pass through the cell membrane of living cells, but could only pass through the disordered area of the dead cell membrane to reach the nucleus, and is embedded in the cell's DNA double helix to produce red fluorescence (Ex=535 nm, Em=617 nm), such that the PI only stains dead cells.

5.2 Instruments and equipment

| | Specification or | |
|---|---|---|
| Instrument Name | model | Manufacturer |
| Laser scanning confocal | 37XB | Shanghai Optical |
| microscope | | Instruments |
| Single-channel manual | 100-1000 μL | ThermoFisher |
| adjustable pipette | | |
| Single-channel manual | 20-200 μL | ThermoFisher |
| adjustable pipette | | |
| Centrifuge | Sorvall ST1 Plus | ThermoFisher |
| Cell counter | AP-0650010 | MARIENFELD |

5.2.1 Reagents

| Reagent name | Cat. No. | Manufacturer |
|---|---|---|
| PBS | AH30026713 | Hyclone |
| Calcein-AM/PI | CA1630 | Solarbio |
| Assaybuffer | EPX-11110-000 | Thermo |

5.2.2 Consumables

| Consumables | Cat. No. | Manufacturer |
|---|---|---|
| 1.5 mL EP tube | 509-GRD-Q | QSP |
| 50 mL centrifuge tube | 339652 | ThermoNunc |
| 15 mL centrifuge tube | 339650 | ThermoNunc |
| 24-well culture plate | 142475 | ThermoNunc |

5.3 Experimental method

5.3.1 Cell seeding on printed product

5.3.1.1 Three samples were selected from each group of freeze-dried printed products and sterilized with ultraviolet irradiation for 30 min.

5.3.1.2 The cells in a T75 culture flask were digested and counted, and a cell suspension was adjusted to have a concentration of 4×10⁴ cells/50 μL.

5.3.1.3 50 μL of the suspension was added dropwise into the center of each print, incubated in a CO₂ incubator at 37° C. 150 μL of the suspension was added every 30 min to keep the printed product moist. After incubation for 1 h, 800 μL of fresh medium was added.

5.3.1.4 On the 1st, 7th, and 14th days after seeding cells, live and dead cells were stained and photographed under a confocal microscope; undetected specimens should be replaced with medium every 2 days to 3 days.

5.3.2 Live and dead cell staining steps

5.3.2.1 10×AssayBuffer was taken out from the low-temperature refrigerator and dissolved at room temperature to obtain a dissolved solution. 1.5 mL of the dissolved solution was added into 13.5 mL of sterilized water to obtain 15 mL of 1×AssayBuffer.

5.3.2.2. Preparation of 1× staining working solution: the Calcein-AM solution and PI solution stored at a relatively low temperature were returned to room temperature for 30 min.

5.3.2.3 0.75 μL of Calcein-AM solution and 2.25 μL of PI solution were added with 1.5 mL of 1×AssayBuffer, and mixed to be uniform. The cell-loaded printed product was removed from the 12-well plate and placed into a new 12-well plate, washed 2 times with 1×AssayBuffer. 500 μL of staining working solution was added to each well and incubated at 37° C. for 30 min.

5.3.2.4 The staining working solution was removed, then added with 1×AssayBuffer to wash 2 times, and the original medium was added to the well plate.

5.3.3 Photographing

5.3.3.1 An FV3000 system was started according to the opening sequence of the laser confocal instrument.

The cell sample was found through the eyepiece. The button “Switch Objective” on the TPC interface was clicked to switch the objective lens, the DIA button was clicked, and the Z-axis position was adjusted to find the focal plane of the cell sample.

5.3.3.2 A detection channel, a green fluorescence channel (Ex=490 nm, Em=515 nm), and a red fluorescence channel (Ex=535 nm, Em-617 nm) were set in the [PMTsetting] tool window.

5.3.3.3 The button “Live” under the [LIVE] tool window was clicked, the focal plane effect was adjusted according to the preview image effect, and desirable laser intensity (%), HV value (V), Gain (X) and other parameters were set.

5.3.3.4 The button “File” in the [Acquire] tool window was clicked, a folder was selected to save the image, the image to be entered was named, and the button “LSM Start” was clicked to start image acquisition.

5.3.3.5 Under a fluorescence microscope, living cells (yellow-green fluorescence) were detected with an excitation filter of 490±10 nm and dead cells (red fluorescence) were detected with an emission filter of 545 nm simultaneously.

5.3.3.6 Photographing was conducted on the live cell filter through four different light source channels (Live, dead, TD (i.e. no fluorescence), and merge) to obtain the staining images of live and dead cells.

VI. In vivo experiment on skull defect repair with hydrogel

6.1 microCT detection of osteogenesis efficiency:

6.1.1 Experimental materials:

1. Experimental animals: SD rats, male, 6-8 Weeks

2. Printed skull flap

3. Surgical instruments

4. Anesthetics

5. microCT scanner

6.1.2 Experimental steps:

1) Preparation before animal surgery: in accordance with experimental animal ethics and operating procedures, all experimental animals and equipment required for surgery were prepared in advance. A skull flap was prepared in advance and injected into the skull defect according to the experimental design.

2) Surgical operation: the experimental animals were anesthetized and fixed on the operating table. The surgical site was prepared by local disinfection and shaving. Preparation of the skull defect model: a standard size defect was created in the rat skull through surgical instruments. The pre-prepared skull flap was filled into the skull defect. The wound was sutured and subsequent treatments such as disinfection and pain relief medication were administered.

3) Postoperative management: the experimental animals were restored to anesthetized awake state and placed in a comfortable environment. The recovery of experimental animals was observed.

4) End of experiment and sampling: the experiment was terminated at a predetermined time point (for example, 2 months after surgery). The animals were sacrificed under CO₂ conditions and the entire skull was removed; the experimental animals were scanned with a micro-CT scanner to obtain a micro-CT result chart showing the effectiveness of skull defect repair in the sample animal experiment, thus evaluating the skull defect repair.

6.2 In vivo effectiveness of skull flap detected by Masson-stained sections 6.2.1

4% Paraformaldehyde (PFA) Decalcification, dehydration, clearing, and embedding reagents

Microtome and tissue slicer

Masson staining kit

Optical microscopes and digital image acquisition systems

6.2.2

1) End of experiment and sampling:

The skull samples in 5.1 were photographed and then fixated with 4% PFA for 12 h to maintain tissue morphology. The skull samples were decalcified, dehydrated, cleared, and embedded.

2) Slicing and staining: the skull samples were cut into 5-20 μm slices with a microtome and tissue microtome, and the slices each were fixed on glass slides, and subjected to Masson staining with a Masson staining kit to obtain a Masson staining rendering of the sample, thus observing the tissue structure and distribution of the skull flap. Masson-stained sections were observed using optical microscopy to evaluate tissue structure, inflammatory response, and biocompatibility of the skull flap.

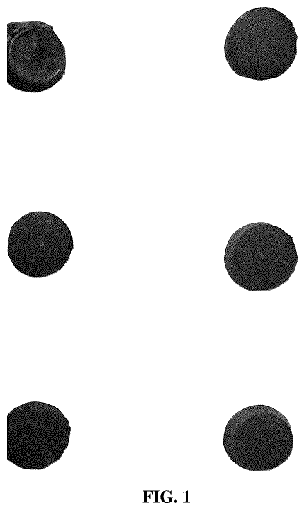

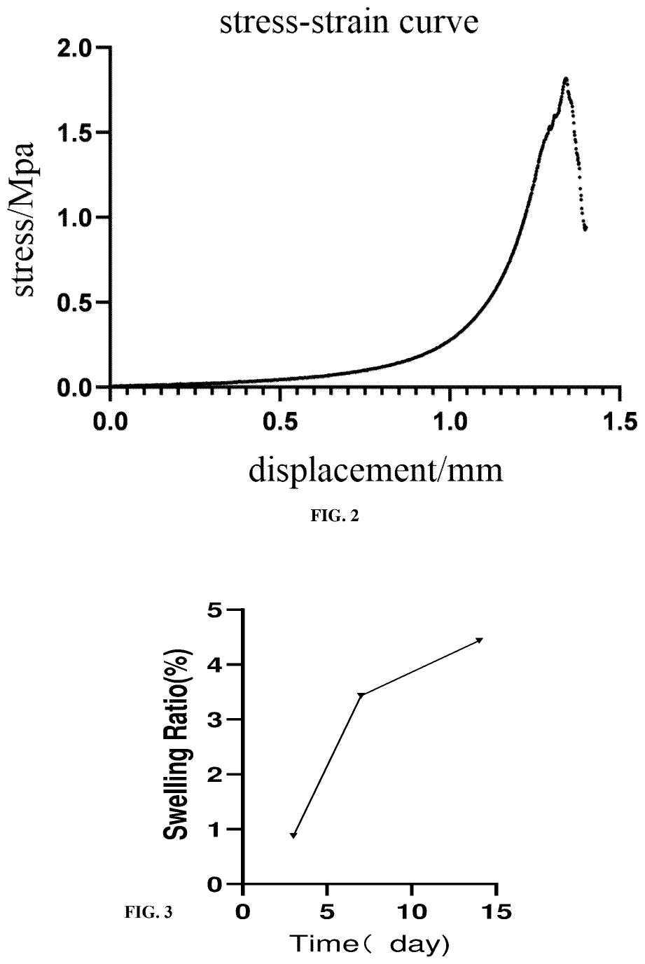

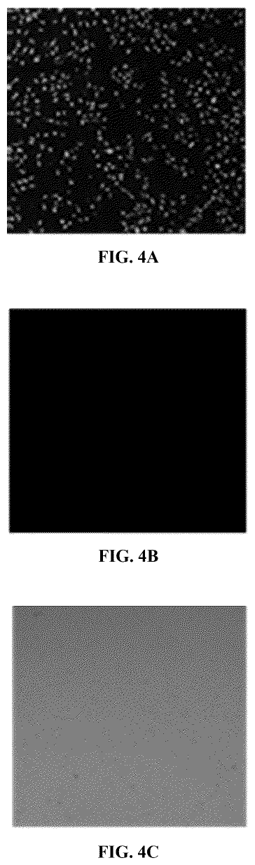

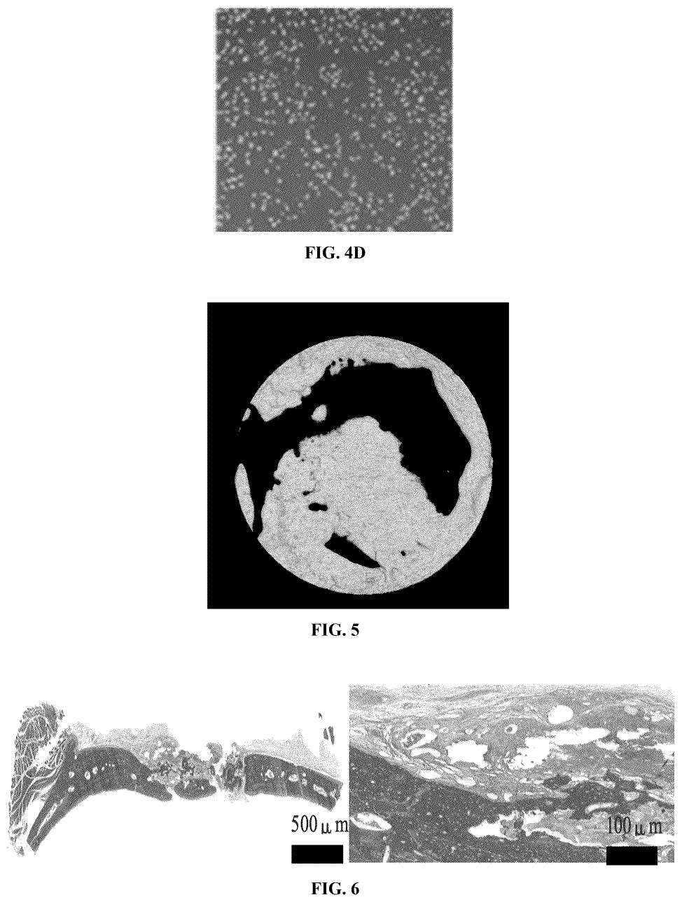

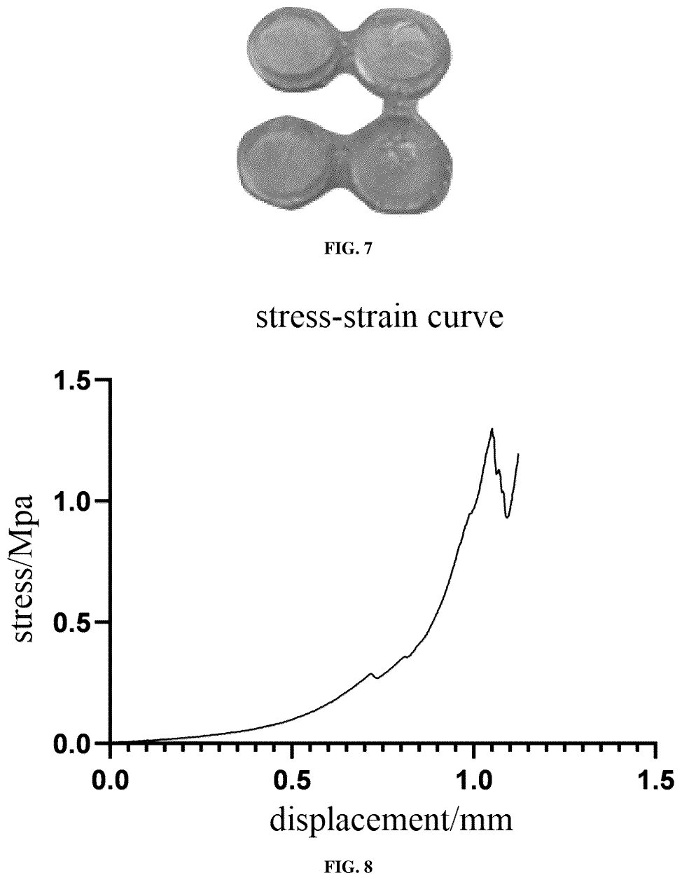

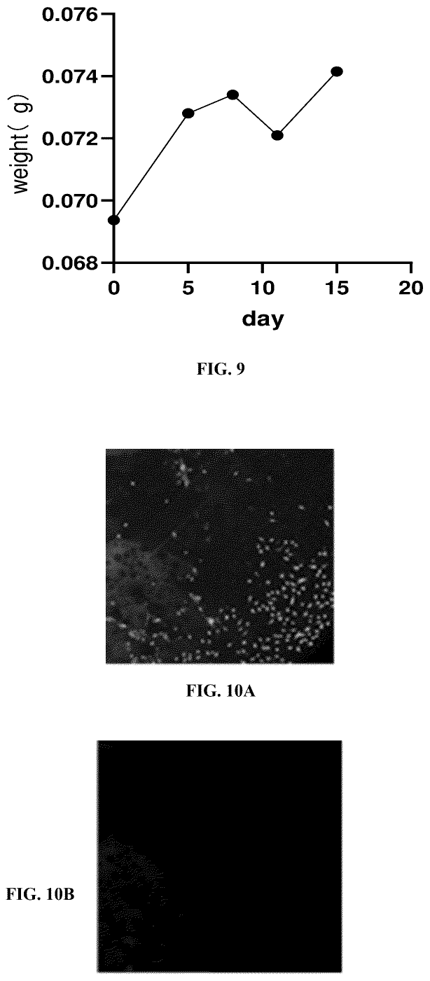

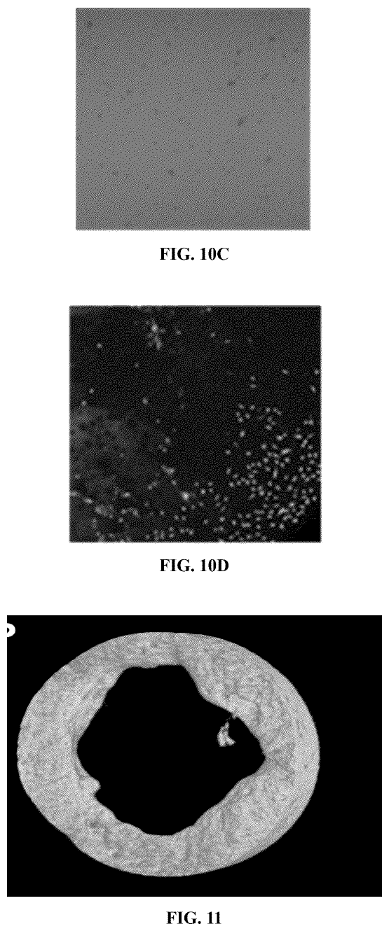

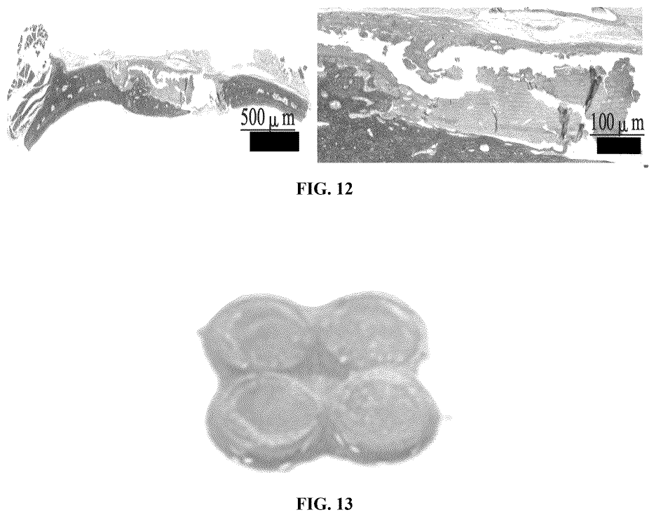

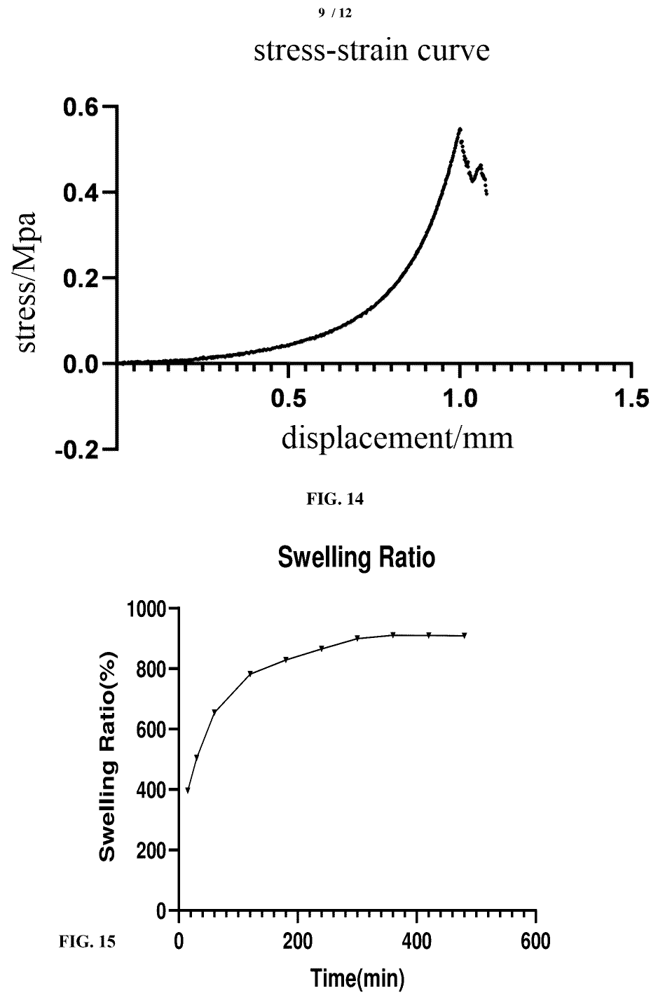

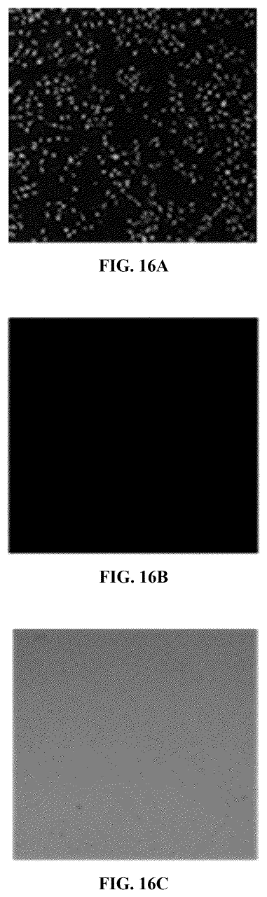

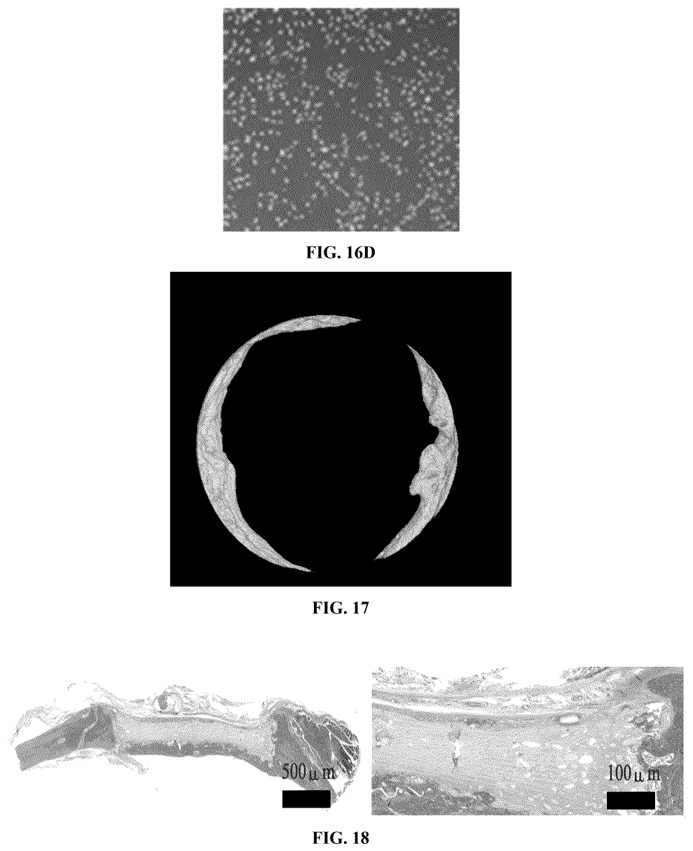

Test results:

| Test items | Example 1 | Comparative Example 1 | Comparative Example 2 |
|---|---|---|---|
| Appearance | Transparent or milky | Transparent or milky | Transparent or milky |
| | white liquid, liquid state | white liquid, liquid state | white liquid, liquid state |
| | at 37° C., milky white | at 37° C., milky white | at 37° C. that was |
| | semi-solid state at 4° C.; | semi-solid state at 4° C.; | relatively viscous, milky |
| | after adding 5 wt % | after adding 5 wt % | white semi-solid state at |
| | photoinitiator and 3 | photoinitiator and 3 wt % | 4° C.; after adding 5 wt % |
| | wt % photoresist to the | photoresist to the bioink | photoinitiator and 3 wt % |
| | bioink precursor, the | precursor, the printing | photoresist to the bioink |
| | printing ink becomes a | ink becomes a yellow | precursor, the printing |
| | yellow transparent | transparent liquid | ink becomes a yellow |
| | liquid | | transparent liquid |
| Printing effect | After printing, the | After printing, the | After printing, the |
| | sample is consistent | sample is consistent with | sample is consistent with |
| | with the preset printing | the preset printing model, | the preset printing model, |
| | model, completely | completely cured, the | completely cured, the |
| | cured, and has clear | printed sample is intact | printed sample is |
| | boundaries. The | and has relatively clear | relatively intact and has |
| | appearance of the | boundaries. The | relatively clear |
| | sample after soaking in | appearance of the sample | boundaries, showing |
| | tea polyphenol is tan, as | after soaking in tea | transparent, as shown in |
| | shown in FIG. 1. | polyphenol is tan, as | FIG. 13. |
| | | shown in FIG. 7. | |
| Mechanical | The sample has a size | The sample has a size of | The sample has a size of |
| properties | of 8 × 8 × 2 mm and a | 8 × 8 × 2 mm and a | 8 × 8 × 2 mm and a |
| | compression | compression | compression |
| | performance of 1.8 | performance of 1.253 | performance of 0.548 |
| | MPa. The mechanical | MPa. The mechanical | MPa. The mechanical |
| | performance test is | performance test is | performance test is |
| | shown in FIG. 2. | shown in FIG. 8. | shown in FIG. 14. |
| Biocompatibility | Referring to FIGs. 4A- | Referring to FIGs. 10A- | Referring to FIGs. 16A- |
| | 4D (relatively desirable | 10D (ordinary cell | 16D (ordinary cell |
| | cell growth status) | growth status) | growth status) |
| In vivo | As shown in FIG. 5, a | As shown in FIG. 11, a | As shown in FIG. 17, a |
| evaluation | large amount of new | small amount of new | small amount of new |
| | bone could be observed | bone production could be | bone could be observed |
| | from CT. | observed from CT. | form CT. |
| | As shown in FIG. 6, a | As shown in FIG. 12, a | As shown in FIG. 18, a |
| | large number of new | small amount of new | small amount of new |
| | collagen blood vessels | collagen blood vessels | collagen blood vessels |
| | could be observed from | could be observed from | could be observed from |
| | Masson staining. | Masson staining. | Masson staining. |
| | During the observation | During the observation | During the observation |
| | period, there were no | period, there were no | period, there were no |
| | abnormalities in the | abnormalities in the | abnormalities in the |
| | weight and living | weight and living | weight and living |
| | conditions of the rats. | conditions of the rats. | conditions of the rats. |

Comparison of the above test results shows that adding GelMA and tea polyphenol could give printed product better mechanical properties and bio-compatibility.

Finally, it should be noted that the above embodiments are merely intended to explain the technical solutions of the present disclosure, rather than to limit the present disclosure. Although the present disclosure is described in detail with reference to the above embodiments, those of ordinary skill in the art should understand that they could still modify the technical solutions described in the above embodiments or make equivalent substitutions on some technical features therein without departing from the concept of the technical solutions, and these modifications or substitutions should fall within the scope of the present disclosure.

What is claimed is:

1. A method for preparing a skull flap by photo-curing 3D (three dimensions) printing, comprising the following steps:
(1) constructing a 3D printing model: generating a corresponding 3D model based on a cranial scan data of a patient;
(2) preparing a 3D printing ink: weighing a photo-crosslinking hydrogel, mixing the photo-crosslinking hydrogel and a nanoclay solution to obtain a mixed system, and then adding a photoinitiator and a photoresist into the mixed system to obtain the 3D printing ink; and
(3) conducting photo-curing 3D printing: subjecting the 3D printing ink to ultrasonic mixing, then filling into an ink tank of a photo-curing 3D printer, transferring the corresponding 3D model into the photo-curing 3D printer, and subjecting the corresponding 3D model to printing to obtain the skull flap;
wherein the nanoclay solution comprises an extracellular matrix component.

2. The method according to claim 1, wherein the cranial scan data of the patient in step (1) is obtained through computed tomography (CT) scanning or magnetic resonance imaging (MRI).

3. The method according to claim 1, wherein the 3D printing ink in step (2) is prepared by weighing 5 wt % to 20 wt % of the photo-crosslinking hydrogel, mixing the 5 wt % to 20 wt % of the photo-crosslinking hydrogel and 1 wt % to 4 wt % of the nanoclay solution to obtain the mixed system, and then adding 0.5 wt % to 5 wt % of the photoinitiator and 0.001 wt % to 3 wt % of the photoresist into the mixed system.

4. The method according to any one of claim 1, wherein the photo-crosslinking hydrogel is at least one selected from the group consisting of methacrylated gelatin (GelMA), methacryloyl sodium alginate (AlgMA), and methacrylated hyaluronic acid (HAMA).

5. The method according to any one of claim 1, wherein a nanoclay in the nanoclay solution is at least one selected from the group consisting of hydroxyapatite, tricalcium phosphate, and lithium magnesium silicate.

6. The method according to claim 1, wherein the extracellular matrix component comprises at least one selected from the group consisting of a calcium phospholipid component, collagen protein, and glycosaminoglycan.

7. The method according to claim 1, wherein the nanoclay solution further comprises a chemokine.

8. The method according to claim 7, wherein the chemokine is at least one selected from the group consisting of a ciliary neurotrophic factor (CNTF) neural factor, a vascular endothelial growth factor (VEGF) vascular factor, and a bone morphogenetic protein 2 (BMP-2) bone repair factor.

9. The method according to claim 1, wherein the ultrasonic mixing in step (3) is conducted at a power of 1,000 W to 1,300 W for 0.5 seconds to 1.5 seconds.

10. The method according to claim 1, wherein the corresponding 3D model in step (3) is cut into multiple two-dimensional layers through model slicing, and then the multiple two-dimensional layers are transferred into the photo-curing 3D printer and subjected to the printing.

11. The method according to any one of claim 3, wherein the photo-crosslinking hydrogel is at least one selected from the group consisting of methacrylated gelatin (GelMA), methacryloyl sodium alginate (AlgMA), and methacrylated hyaluronic acid (HAMA).

12. The method according to any one of claim 3, wherein a nanoclay in the nanoclay solution is at least one selected from the group consisting of hydroxyapatite, tricalcium phosphate, and lithium magnesium silicate.
